(19) United States
(12) Patent Application Publication
(10) Pub. No.: US 2025/0000692 A1
(43) Pub. Date: Jan. 2, 2025

(54) HEATING BREASTPLATE AND CONTROL SYSTEM FOR PREVENTING ACCIDENTAL HYPOTHERMIA

(71) Applicant: Thermo-Trauma, Briançon (FR)

(72) Inventors: Thomas SPADONI, Puy Saint Andre (FR); Rémy BERGON, Briançon (FR)

(73) Assignee: Thermo-Trauma, Briançon (FR)

(21) Appl. No.: 18/345,032

(22) Filed: Jun. 30, 2023

(51) Int. Cl. A61F 7/02; A61F 7/00

(52) U.S. Cl. CPC A61F 7/02; A61F 7/007; A61F 2007/0019; A61F 2007/0078; A61F 2007/0234

(57) ABSTRACT

Provided is a system for controlling temperature of a heating breastplate placed on a body of an injured patient, including a heating element positioned within a heating breastplate, the heating element comprising a positive temperature coefficient, the heating element connected to one or more batteries positioned within the heating breastplate; and a controller connected to the one or more batteries and the heating element to regulate a temperature of a body of an injured patient, wherein the controller is programmed or configured to regulate the temperature of the heating element to provide a target temperature value of the heating element, such that heating element provides a uniform and constant temperature to the body of the injured patient.

### BACKGROUND

### Field

The present invention relates to a heating breastplate and associated control system providing embodiments to fight accidental hypothermia of an injured person and/or patient by stabilizing or raising an injured patient's body temperature, in particular in a pre-hospital phase.

### Technical Background

Accidental hypothermia is a frequent and serious clinical situation that may occur in multiple trauma injuries and can affect 30% to 50% of patients suffering from major trauma. This is described in particular in the study by Søreide K., entitled Clinical and translational aspects of hypothermia in major trauma patients: from pathophysiology to prevention, prognosis and potential preservation and published in the scientific journal Injury in 2014.

In addition, the consequences of such hypothermia on bleeding disorders are responsible for nearly 50% of deaths within 48 hours of admission to intensive care, as detailed in the following studies:

Brown D. J., Brugger H., Boyd J., Paal P., Accidental hypothermia, N Engl J Med, November 2012; and

Silfvast T., Pettila V., Outcome from severe accidental hypothermia in Southern Finland—a 10-year review, Resuscitation, December 2003.

Management of accidental hypothermia is a major challenge in emergency medicine. To date, the systems available on the market to support accidental hypothermia include passive, insulating systems, that function by avoiding heat loss. Examples of available systems include survival blankets (Comed type survival blanket) and other types of active warming and/or insulation systems.

Some systems may include blankets using energy of a chemical reaction such as heaters releasing heat when they are struck. “Ready-Heat® II Torso Blanket” is an example of a blanket system using a chemical reaction to produce heat. Such systems are more efficient than passive warming systems, but such systems are bulky and must be disposed of after use (e.g., not reusable).

There are other active heating systems that may use electrical energy. For example, a system known as “UniqueResc+®” from the company “Geratherm®.” Such systems operate using a 150 W, 240V AC power adapter that must be plugged into a wall outlet during use or external batteries that may need to be replaced often, complicating the use of these systems in the field. There are often no available wall outlets in the field, and some rescue scenarios may require a generator to power such systems. Generators can be cumbersome to transport to many rescue and/or injury sites. Batteries may run out of available power unexpectedly in the field, and rescue crews must make sure to always have back-up batteries available. Batteries and other electrical equipment may weigh down and/or be positioned awkwardly on available systems, causing discomfort for the patient. Other factors such as weather at a rescue site may affect the utility and/or integrity of electrical systems.

In addition, such active warming systems have a size and a bulk that can make their transport difficult. These systems are not designed with an ergonomic focus to contact a patient's body with a close, ergonomic fit (e.g., contact as close as possible to the patient's body). The positioning of available active warming systems often embarrasses first aid responders. Indeed, such active warming systems are mostly positioned outside the clothes of an injured person, making it difficult for the heat to diffuse throughout the patient's body. Further, these systems do not apply heat directly to a patient's body, as close as possible to the body.

### SUMMARY

The present invention aims to remedy the aforementioned drawbacks, in particular to propose a control system for a heating breastplate, being fixed at the level of the neck of an injured patient by a neckline and ergonomically fitting the neck and the trunk of the injured patient (e.g., the thorax, the abdomen, and sides).

Indeed, in order to prevent or treat accidental hypothermia, disclosed embodiments aim to warm the injured patient at a level of the trunk, where the blood accumulates to protect the noble organs as well as the areas where the vessels are superficial such as by example in the carotid region, in other words at the level of the neck, allowing a warming of blood flowing to the brain of the injured patient. In addition to the effects on the body temperature of an injured patient, the installation of a heat source on the trunk of an injured patient has a reassuring effect, improves the comfort of the injured patient and reduces stress, facilitating the intervention of rescuers.

Embodiments of a heating breastplate include a control system that may be rechargeable, for example, by using rechargeable batteries. The use of rechargeable batteries ensures no wall outlets or generators are required for use in the field, and the batteries may be easily recharged. The batteries may be located on and/or within the heating breastplate such that they provide for comfort of the injured patient, and so the batteries weigh the heating breastplate downward to conform to the injured patient's body ergonomically.

Embodiments of the control system for the heating breastplate may also allow for control of heating of the breastplate, such that an injured patient does not get too hot and is warmed to an appropriate temperature, while embodiments also provide for adequate heating of the batteries in colder weather locations. This may ensure that the batteries do not become too cold such that the batteries lose power or cannot adequately supply power to the heating breastplate.

Some embodiments may provide a system for controlling temperature of a heating breastplate placed on a body of an injured patient. The system may include a heating element positioned within a heating breastplate. The heating element may include a positive temperature coefficient. The heating element may be connected to one or more batteries positioned within the heating breastplate. The system may include a controller connected to the one or more batteries and the heating element to regulate a temperature of a body of an injured patient. The controller may be programmed or configured to regulate the temperature of the heating element to provide a target temperature value of the heating element, such that heating element provides a uniform and constant temperature to the body of the injured patient.

In some embodiments, when regulating the temperature of the heating element, the controller may be further programmed or configured to: determine a first temperature value of the heating element based on a resistance of the heating element. The controller may be further programmed or configured to compare the first temperature value of the heating element to a temperature threshold. The controller may be further programmed or configured to regulate the temperature of the heating element to the target temperature by increasing or decreasing the temperature of the heating element based on comparing the first temperature value of the heating element to the temperature threshold.

In some embodiments, determining a first temperature value of the heating element based on a resistance of the heating element may include measuring the resistance of the heating element to provide a resistance value.

In some embodiments, determining the first temperature value of the heating element based on the resistance of the heating element may include determining the first temperature value of the heating element by comparing the resistance value to a reference relationship of resistance and temperature based on a positive temperature coefficient.

In some embodiments, the one or more batteries may be configured to connect to a battery charger while remaining positioned within the heating breastplate.

In some embodiments, the heating breastplate may be placed on the body of the injured patient.

In some embodiments, the temperature threshold may be set by a user of the heating breastplate.

In some embodiments, the controller of the heating breastplate may include a minimum temperature threshold such that the controller may regulate the temperature of the heating element to maintain a temperature of the one or more batteries above the minimum temperature threshold.

In some embodiments, the system may further include a display positioned on the heating breastplate. The display may be configured to display a level of charge of the one or more batteries and a time associated with a duration of use of the one or more batteries.

In some embodiments, the heating breastplate may be water resistant.

Some embodiments may provide a heating breastplate intended to be placed on the body of an injured patient. The heating breastplate may include a neckline and a lower part respectively hugging the neckline for the neck and the thorax and the abdomen for the lower part of the heating breastplate. The heating breastplate may include one or more heating elements arranged and positioned within the heating breastplate to heat the thorax, abdomen, and neck of the injured patient after appropriate application of the heating breastplate to the body of the injured patient.

In some embodiments, the neckline and the lower part of the heating breastplate are arranged, by their respective shapes and dimensions, to be positioned and adjusted on the body of the injured patient, from the front face of the body and without mobilizing the injured patient.

In some embodiments, the heating breastplate may include one or more batteries connected to the one or more heating elements to supply electrical energy to the one or more heating elements.

In some embodiments, a lower third of the heating breastplate may include one or more pockets configured to receive the one or more batteries such that the heating breastplate is easier to deploy on the injured patient and such that the weight of the one or more batteries assists in holding the heating breastplate on the injured patient's body by gravity.

In some embodiments, the heating breastplate may further include a controller connected to the one or more batteries and the heating element to regulate a temperature of the body of the injured patient. The controller may be programmed or configured to regulate the temperature of the heating element to provide a target temperature value of the heating element, such that the heating element provides a uniform and constant temperature to the body of the injured patient.

In some embodiments, the one or more batteries may be configured to connect to a battery charger while remaining positioned within the heating breastplate.

Some embodiments may provide a heating breastplate intended to be placed on a body of an injured patient. The heating breastplate may include a neckline and a lower part matching the body of the injured patient respectively at the level of the neck and trunk of the injured patient and a heating element for warming the body of the injured patient. The heating element may be positioned in the neckline and at the level of the lower part of the heating breastplate.

In some embodiments, the neckline may be rectangular in shape and may have a width of seven (7) centimeters and a length of thirty-eight (38) centimeters. The neckline may be arranged to hug the neck of the injured patient.

In some embodiments, the neckline may include a fastening system adding an additional length to the neckline and making it possible to attach the heating breastplate to the injured patient's neck.

In some embodiments, the heating element may include at least one flexible heating resistor formed of electrical conductors powered by batteries. The at least one flexible heating resistor may be configured to transform electrical energy into thermal energy.

In some embodiments, the heating breastplate may include a multilayer textile including a first and a second outer layer of polyamide coated with a polyurethane polymer and enclosing a heating layer. The first outer layer may correspond to a front face of the heating breastplate. The first outer layer may be in contact with an external environment. The second outer layer may correspond to a rear face of the heating breastplate. The second outer layer may be in contact with the body of the injured patient.

In some embodiments, the heating layer may include a printed circuit including a flexible polyester substrate covered with a flexible conductive layer of aluminum. The at least one heating resistor may be etched on the aluminum layer.

In some embodiments, the printed circuit may be covered with a protective layer of polyester.

In some embodiments, the multilayer textile may include a thermal insulation layer between the first outer layer and the heating layer. The thermal insulation layer may include a laminate of cellulose and aluminum.

In some embodiments, the heating breastplate may include pockets at a level of the lower third of the heating breastplate and distributed symmetrically on either side of a center line (M) of the heating breastplate. The pockets may be configured to receive the batteries.

In some embodiments, the heating breastplate may include a storage pocket having a closure system, a storage bag of the heating breastplate and one or more power cables for batteries. The storage pocket may be positioned on a lower part of the heating breastplate and may be configured to contain the storage bag and the power cables.

### BRIEF DESCRIPTION OF THE DRAWINGS

Embodiments of the disclosed subject matter will be better understood and other characteristics and advantages thereof will appear on reading the following description of particular embodiments given by way of illustrative and non-limiting examples and with reference to the appended drawings, among which:

FIG. 1 shows a schematic diagram of a system 100 for controlling temperature of a heating breastplate placed on a body of an injured patient in accordance with non-limiting embodiments of the disclosed subject matter;

FIG. 2 shows a view of a rear face of a heating breastplate according to non-limiting embodiments of the disclosed subject matter;

FIG. 3 shows a heating breastplate in use on a body of an injured patient according to non-limiting embodiments of the disclosed subject matter;

FIG. 4 shows a view of a front face of a heating breastplate according to non-limiting embodiments of the disclosed subject matter;

FIG. 5 shows a schematic view of a heating breastplate according to section AA of FIG. 2 in accordance with non-limiting embodiments of the disclosed subject matter;

FIG. 6 shows a schematic sectional view of a heating breastplate according to non-limiting embodiments of the disclosed subject matter;

FIG. 7 shows an embodiment of a heating element of the heating faceplate in accordance with non-limiting embodiments of the disclosed subject matter;

FIG. 8 shows a flow chart of a method for updating a temperature of a heating element of a heating breastplate according to non-limiting embodiments of the disclosed subject matter; and

FIG. 9 shows a flow chart of a method for automatically protecting a temperature of one or more batteries of a heating breastplate according to non-limiting embodiments of the disclosed subject matter.

### DETAILED DESCRIPTION

In order to simplify the description, the same reference is used in different figures to designate the same object. Thus, when the description cites a referenced object, this object may be identified in several figures. Furthermore, the figures as well as the description are given by way of non-limiting embodiments.

In the present disclosure, the term “rear face” corresponds to the face of a heating breastplate coming into contact with a body of an injured patient and the term “front face” corresponds to the face of the heating breastplate being in contact with an external environment when the heating breastplate is positioned on an injured patient.

Embodiments of a system 100 for controlling temperature of a heating breastplate placed on a body of an injured patient may include an electronics assembly 110 to provide power to a heating breastplate 200 to produce heat (e.g., to convert electrical energy into thermal energy) via a heating element 130 arranged in the heating breastplate 200. The electronics assembly 110 may be configured to automatically control a temperature of the heating element 130 such that a patient with the heating breastplate 200 placed on a body 300 of the patient is not injured due to the heat of the heating element 130 and/or a temperature of the heating breastplate 200.

Additionally, the electronics assembly 100 may be configured to automatically control the temperature of the heating element 130 such that one or more batteries 106 arranged within the heating breastplate 200 (e.g., in a pocket of the heating breastplate 200) may be maintained above a threshold temperature so the one or more batteries 106 may maintain power and so the one or more batteries 106 do not decrease in stored power due to colder temperatures (e.g., about −5° C.) of an external environment.

Embodiments may include a heating breastplate 200 including a heating element 130. The heating breastplate 200 may be intended to be placed on a body 300 of an injured patient. For example, the heating breastplate 200 may be intended to be placed on a torso of the injured patient. Such a heating breastplate 200 thus makes it possible to treat an injured patient in accidental hypothermia and/or to prevent any hypothermia of an injured patient in an emergency and/or first aid situation.

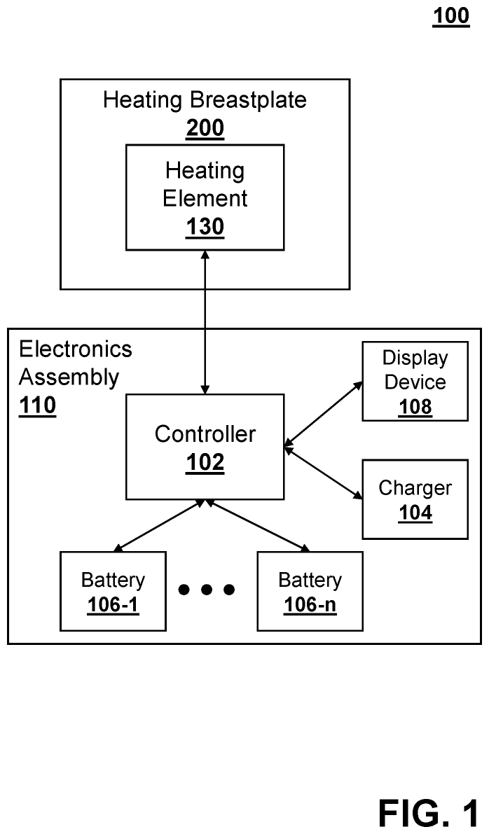

Referring to FIG. 1, FIG. 1 shows a schematic diagram of a system 100 for controlling temperature of a heating breastplate placed on a body of an injured patient in accordance with non-limiting embodiments of the disclosed subject matter. System 100 may include electronics assembly 110, heating element 130, and heating breastplate 200. Electronics assembly 110 may include one or more components, such as controller 102, charger 104, and battery 106-1 through battery 106-n (referred to collectively as batteries 106 and individually as battery 106, where appropriate). In some embodiments, electronics assembly 110 may include a single component. Alternatively, electronics assembly may include various separate components.

As shown in FIG. 1, controller 102 may include at least one processor (e.g., a multi-core processor), such as a central processing unit (CPU), an accelerated processing unit (APU), a graphics processing unit (GPU), a microprocessor, and/or the like. In some embodiments, controller 102 may be programmed or configured to perform one or more steps of methods and/or processes described herein. In some embodiments, controller 102 may include one or more processors executing instructions (e.g., software instructions) that cause controller 102 to perform one or more steps of methods and/or processes described herein. In some embodiments, controller 102 may be in communication with heating element 130, charger 104, batteries 106 (e.g., battery 106-1 to battery 106-n), and/or display 108. In some embodiments, controller 102 may be capable of receiving information (e.g., data, and/or the like) from and/or transmitting information to heating element 130, charger 104, batteries 106, and/or display 108.

Controller 102 may be configured with any combination of one or more processors, attendant local and/or remote memory, and software instructions, each processor being configured to execute software functionality as stored in local or remote memory to perform operational features as described herein. A hardware controller, as used herein, can be a special purpose or a general purpose controller and/or processor device. The hardware controller device may be connected to a communications infrastructure, such as a bus, message queue, network, multi-core message-passing scheme, etc. An exemplary controller, as used herein, can also include a memory (e.g., random access memory, read-only memory, etc.), and can also include one or more additional memories. The memory and the one or more additional memories may be read from and/or written to in a well-known manner. In exemplary embodiments, the memory and one or more additional memories may be non-transitory computer readable recording media.

Any of the controllers disclosed herein can be part of or in communication with a machine (e.g., a computer device, a logic device, a circuit, an operating module (hardware, software, and/or firmware), etc.). The controller can be hardware (e.g., processor, integrated circuit, central processing unit, microprocessor, core processor, computer device, microcontroller, etc.), firmware, software, etc. configured to perform operations by execution of instructions embodied in computer program code, algorithms, program logic, control, logic, data processing program logic, artificial intelligence programming, machine learning programming, artificial neural network programming, automated reasoning programming, etc. The processor can receive, process, and/or store data related to sensors and/or network communications, for example.

Any of the controllers disclosed herein may include a scalable processor, a parallelizable processor, a multi-thread processing processor, etc. For example, controller 102 may include any integrated circuit or other electronic device (or collection of devices) capable of performing an operation on at least one instruction, which can include a Reduced Instruction Set Core (RISC) processor, a CISC microprocessor, a Microcontroller Unit (MCU), a CISC-based Central Processing Unit (CPU), a Digital Signal Processor (DSP), a Graphics Processing Unit (GPU), a Field Programmable Gate Array (FPGA), a system on a chip (SoC), etc. The hardware of such devices may be integrated onto a single substrate (e.g., silicon “die”), or distributed among two or more substrates. Various functional aspects of controller 102 may be implemented solely as software or firmware associated with the controller 102.

Controller 102 may include one or more processing or operating modules. A processing or operating module can be a software or firmware operating module configured to implement any of the functions disclosed herein. The processing or operating module may be embodied as software and stored in memory, the memory being operatively associated with the processor and/or controller. A processing module can be embodied as a web application, a desktop application, a console application, an embedded application, etc.

Controller 102 may include or be associated with a computer or machine readable medium. The computer or machine readable medium may include memory. Any of the memory discussed herein can be computer readable memory configured to store data. The memory may include a volatile or non-volatile, transitory or non-transitory memory, and may be embodied as an in-memory, an active memory, a cloud memory, etc. Examples of memory can include flash memory, Random Access Memory (RAM), Read Only Memory (ROM), Programmable Read only Memory (PROM), Erasable Programmable Read only Memory (EPROM), Electronically Erasable Programmable Read only Memory (EEPROM), FLASH-EPROM, Compact Disc (CD)-ROM, Digital Optical Disc DVD), optical storage, optical medium, a carrier wave, magnetic cassettes, magnetic tape, magnetic disk storage or other magnetic storage devices, or any other medium which can be used to store the desired information and which can accessed by a processor and/or controller 102.

Controller 102 may be in communication with other processors and/or controllers of other devices (e.g., a computing device, a computer system, a laptop computer, a desktop computer, etc.). For instance, controller 102 may be in communication with a separate controller for charger 104, batteries 106, heating element 130, display 108, and/or the like. Controller 102 may have transceivers or other communication devices and/or circuitry to facilitate transmission and reception of wireless signals. Controller 102 may include an API as a software intermediary that allows two or more applications to talk to each other. Use of an API may allow software of controller 102 to communicate with software of another processor and/or controller of another device.

The memory of controller 102 may include a non-transitory computer-readable medium. The term “computer-readable medium” (or “machine-readable medium”) as used herein is an extensible term that refers to any medium or any memory, that participates in providing instructions to controller 102 for execution, or any mechanism for storing or transmitting information in a form readable by a machine (e.g., a computer). Such a medium may store computer-executable instructions to be executed by a processing element and/or control logic, and data which is manipulated by a processing element and/or control logic, and may take many forms, including but not limited to, non-volatile medium, volatile medium, transmission media, etc. The computer or machine readable medium can be configured to store one or more instructions thereon. The instructions can be in the form of algorithms, program logic, etc. that cause controller 102 to execute any of the functions disclosed herein.

Embodiments of the memory may include a processor module and other circuitry to allow for the transfer of data to and from the memory, which can include to and from other components of a communication system. This transfer can be via hardwire or wireless transmission. The communication system can include transceivers, which can be used in combination with switches, receivers, transmitters, routers, gateways, wave-guides, etc. to facilitate communications via a communication approach or protocol for controlled and coordinated signal transmission and processing to any other component or combination of components of the communication system. The transmission can be via a communication link. The communication link can be electronic-based, optical-based, opto-electronic-based, quantum-based, etc. Communications can be via Bluetooth, near field communications, cellular communications, telemetry communications, Internet communications, etc.

Data stored in controller 102 (e.g., in the memory) may be stored on any type of suitable computer readable media, such as optical storage (e.g., a compact disc, digital versatile disc, Blu-ray disc, etc.), magnetic tape storage (e.g., a hard disk drive), or solid-state drive. An operating system may also be stored in the memory.

In an exemplary embodiment, the data can be configured in any type of suitable database configuration, such as a relational database, a structured query language (SQL) database, a distributed database, an object database, etc. Suitable configurations and storage types will be apparent to persons having skill in the relevant art.

Controller 102 may also include a communications interface. The communications interface can be configured to allow software and data to be transferred between controller 102 and external devices. Exemplary communications interfaces can include a modem, a network interface (e.g., an Ethernet card), a communications port, a PCMCIA slot and card, etc. Software and data transferred via the communications interface can be in the form of signals, which can be electronic, electromagnetic, optical, or other signals as will be apparent to persons having skill in the relevant art. The signals can travel via a communications path, which can be configured to carry the signals and can be implemented using wire, cable, fiber optics, a phone line, a cellular phone link, a radio frequency link, etc. Transmission of data and signals can be via transmission media. Transmission media can include coaxial cables, copper wire, fiber optics, etc. Transmission media can also take the form of acoustic or light waves, such as those generated during radio-wave and infrared data communications, or other form of propagated signals (e.g., carrier waves, digital signals, etc.).

Memory semiconductors (e.g., DRAMs, etc.) can be means for providing software to controller 102. Computer programs (e.g., computer control logic) can be stored in the memory. Computer programs can also be received via the communications interface. Such computer programs, when executed, can enable controller 102 to implement the methods and/or processes as described herein. In particular, the computer programs stored on a non-transitory computer-readable medium, when executed, may enable controller 102 to implement the methods and/or processes as described herein. Accordingly, such computer programs may represent controllers 102 for a computing device.

In some embodiments, controller 102 may include batteries 106 and display 108 in a single assembly. Alternatively, controller 102, batteries 106, and display 108 may be contained in separate (e.g., modular) components. Controller 102 may be connected to charger 104 (e.g., when charging batteries 106). Alternatively, charger 104 may be directly connected to batteries 106. In some embodiments, controller 102 may be connected to batteries 106 and/or display 108.

As further shown in FIG. 1, charger 104 may include a standard charger for a lithium-ion battery, such as a lithium-ion charging pack rated from 12 volts to 14.8 volts including a suitable 110 volt-240 volt AC connector and/or a 3.7 volt to 14.8 volt DC connector. Other suitable chargers may be used depending on the type of battery.

Battery 106 may include a 14.8 volt, 3.7 ampere battery, such as a lithium-ion battery. Batteries 106 may be rated for 40 watts of power at 20° C. (e.g., in an external environment). In some embodiments, each battery 106 may have at least two cells, and in some embodiments may have at least 8 cells. In some embodiments, each battery 106 may have at least 4 packs (e.g., 8 cells) with 2 cells per pack so that batteries 106 may be arranged in heating breastplate 200 so as to not be mechanically linked such that batteries 106 conform to heating breastplate 200 and a body 300 of a patient when heating breastplate 200 is worn by a patient. In some embodiments, batteries 106 may be designed such that they cannot injure a patient when arranged in heating breastplate 200 worn by the patient. In some embodiments, battery 106 may refer to a single battery pack (e.g., 2 battery cells). In other embodiments, battery 106 may refer to four packs (e.g., 8 battery cells total) depending on an arrangement of batteries 106 in heating breastplate 200. In some embodiments, batteries 106 may be configured to connect to a battery charger (e.g., charger 104) while remaining positioned within heating breastplate 200.

Display device 108 may include one or more graphical user interfaces to display on display device 108. For example, display device 108 may display a graphical user interface. Display device 108 may include any computing device, and/or may be included in another computing device such as, but not limited to, a cell phone, a server computer, a desktop computer, a notebook, a laptop computer, a tablet computer, a handheld device, a smart-phone, a thin client, or any other electronic device or computing system capable of receiving display signals from another computing device, such as controller 102, and outputting those display signals to a display device such as, but not limited to, an LCD screen, plasma screen, LED screen, DLP screen, CRT screen, etc. In some embodiments, display device 108 may include a standalone display device that displays signals and/or data received from controller 102.

Heating breastplate 200 may include one or more heating elements 130 to heat heating breastplate 200. Heating breastplate 200 (e.g., heating element 130 thereof) may be connected to controller 102 for controlling a temperature of heating element 130 and for providing power to heating element 130 via batteries 106. In some embodiments, heating breastplate 200 may include display 108 located on a front face of heating breastplate 200. Alternatively, display 108 may be located on electronics assembly 110 (e.g., on controller 102).

In some embodiments, heating element 130 may be positioned within heating breastplate 200 (e.g., embedded in material of heating breastplate 200). In some embodiments, heating element 130 may include a material having a positive temperature coefficient. Heating element 130 may be connected to (e.g., via one or more wires) one or more batteries 106 positioned on or within heating breastplate 200 (e.g., lower part 220).

The number and arrangement of systems and devices shown in FIG. 1 are provided as an example. There may be additional systems and/or devices, fewer systems and/or devices, different systems and/or devices, and/or differently arranged systems and/or devices than those shown in FIG. 1. Furthermore, two or more systems or devices shown in FIG. 1 may be implemented within a single system or device, or a single system or device shown in FIG. 1 may be implemented as multiple, distributed systems or devices. Additionally or alternatively, a set of systems (e.g., one or more systems) or a set of devices (e.g., one or more devices) of system 100 may perform one or more functions described as being performed by another set of systems or another set of devices of system 100.

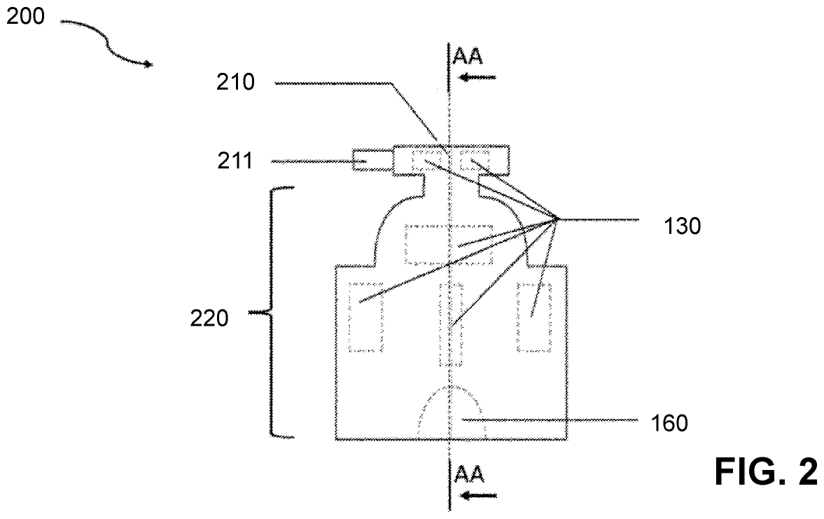

Referring now to FIG. 2, heating breastplate 200 according to non-limiting embodiments may include a neckline 210 intended to hug a neck of the injured patient and a lower part 220 intended to hug a trunk (e.g., lower torso) of an injured patient. In some embodiments, the neckline 210 and the lower part 220 both may have at least one heating element 130, warming a body 300 of the injured patient.

The neckline 210 is a piece of textile of rectangular shape arranged to adapt and/or conform to a neck of an injured patient, regardless of a morphology of the injured patient. In some embodiments, neckline 210 may have dimensions of between three (3) and fifteen (15) centimeters in width and between thirty (30) and fifty (50) centimeters in length. In some embodiments, the width of neckline 210 may be seven (7) centimeters and the length of neckline 210 may be thirty-eight (38) centimeters to correspond to all patient morphologies.

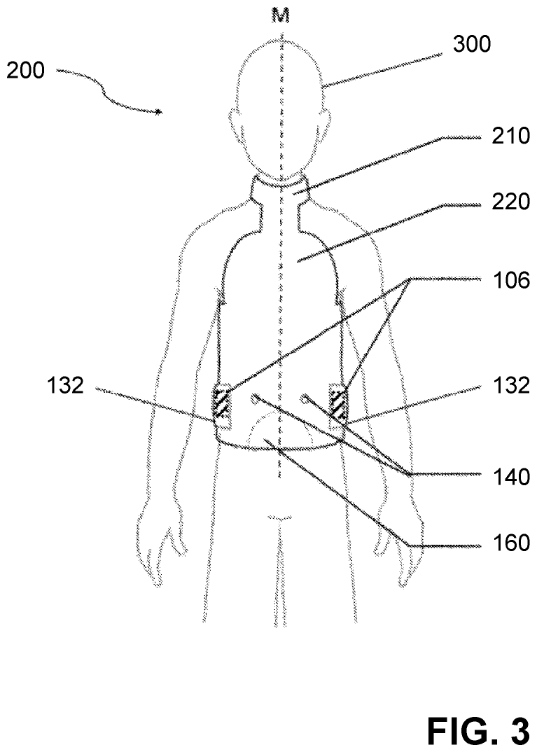

In some embodiments, neckline 210 may include fastening system 211 making it possible to fix the heating breastplate 200 to the neck of the injured patient as illustrated in FIG. 3. Indeed, fastening system 211 adds an additional length to neckline 210 and thus makes it possible to surround an entire neck of the injured patient. Fastening system 211 can be made in different ways, such as, for example, a hook-and-loop fastening system or otherwise known as “scratches” of the type known under the “Velcro®” brand. Any other type of system allowing fastening system 211 to be quickly attached to the injured patient's neck can be used, such as for example a zipper or press-stud system, or other similar devices.

In some embodiments, when fastening system 211 is attached around the injured patient's neck, heating breastplate 200 can be easily unrolled over the rest of body 300 by pulling lower part 220 downwards, thus avoiding positioning and adjustment difficulties on body 300. Lower part 220 then may be positioned naturally on a torso of the injured patient, conforming to a thorax of the injured patient, an abdomen and flanks. In this way, it may be possible to position heating breastplate 200 without mobilizing the injured patient. In some embodiments, a cardiac massage may be performed on the injured patient without having to remove heating breastplate 200. In this way, heating breastplate 200 may be easily positioned for various procedures without moving the injured patient.

In some embodiments, heating breastplate 200 may also be easily removed from body 300 without mobilizing and/or harming the injured patient, facilitating access to the thorax for a nursing staff. For example, heating breastplate 200 may be easily removed without mobilizing the injured patient when intubation of the injured patient is required and/or where a heart rate monitoring device with an electrode for medical monitoring and/or diagnosis must be placed on body 300 of the injured patient. Thus, heating breastplate 200 offers flexibility for procedures to be performed on an injured patient while the heating breastplate is positioned on the injured patient, or, alternatively, heating breastplate 200 may be easily removed when certain procedures must be performed on the injured patient (e.g., intubation, positioning electrodes directly on skin of the injured patient, and/or the like).

FIG. 2 shows heating breastplate 200 with various heating elements 130 which may, for example, include flexible heating resistors 130 distributed over strategic areas of neckline 210 and trunk (thorax, abdomen, flanks—e.g., lower part 220). However, at least one heating element 130, including, for example, a heating resistor 130, may suffice. Heating resistors 130 may be formed of long and/or thin electrical conductors providing a high electrical resistance. Circulation of a current in the electrical conductors may transform electrical energy into thermal energy by the Joule effect.

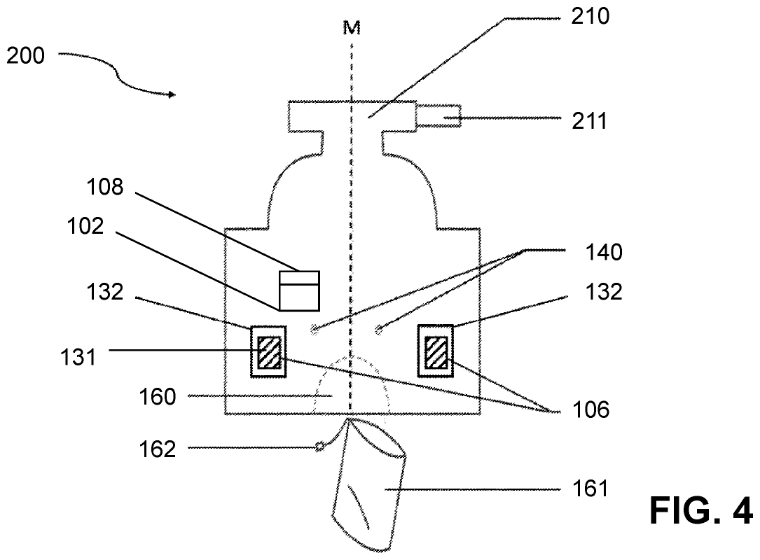

In some embodiments, the electrical conductors may be powered by one or more batteries 106, such as illustrated in FIGS. 3 and 4. Batteries 131 may include lithium-Ion batteries or any other suitable type of battery capable of providing an electrical supply to controller 102, heating elements 130, and/or heating resistors 130. Life of batteries 106 may include at least 4 hours in order to allow emergency services time necessary to reach a hospital from a location of an accident of an injured patient. Thus, heating element 130 powered by batteries 106 integrated into heating breastplate 200 makes it possible to provide rescuers an autonomous device without requiring any other connectors or external power sources necessary for the time of the emergency intervention and/or rescue. In some embodiments, batteries 106 may be equipped with a protection system against short circuits, overheating, and high voltage in order to avoid any risk of burning the injured patient.

Batteries 106, as shown in FIGS. 3 and 4, may be positioned in battery pockets 132. Battery pockets 132 may be located at a level of lower part 220 of heating breastplate 200. For example, battery pockets 132 may be located at a level of the lower third of heating breastplate 200 and distributed symmetrically on either side of a midline M of body 300 of the injured patient. Battery pockets 132 may have quick openings, for example of the self-gripping type or otherwise known as “scratches”, facilitating the accessibility of batteries 106 in the event batteries 106 need to be replaced. Positioning of batteries 106 may make it possible, by a weight of batteries 106, to unfold the heating breastplate 200 entirely downwards by gravity, subsequently facilitating the positioning and maintenance of a position of heating breastplate 200 on body 300 of the injured patient. For example, batteries 106 and battery pockets 132 may be positioned at a level of the flanks of the injured patient on lower part 220. In some embodiments, batteries 106 may be configured to connect to a battery charger (e.g., charger 104, power cables 162, etc.) while remaining positioned within battery pockets 132 of heating breastplate 200.

Heating elements 130 may be activated via on/off power buttons 140, as illustrated in FIGS. 3 and 4. Power buttons 140 may be sealed and fixed to lower part 220 of heating breastplate 200 in order to be easily accessible for powering up heating elements 130 and so as not to interfere with first aid actions.

In some embodiments, heating breastplate 200 may include at least one storage pocket 160 having a quick-closing system among those disclosed herein. Storage pocket 160 may be located in lower part 220 of heating breastplate 200. Storage pocket 160 may contain storage bag 161 of heating breastplate 200 as well as power cables 162 used to connect charger 104 and/or batteries 106 to a power source. In some embodiments, storage bag 161 may be waterproof and may be supplied as a separate component.

In some embodiments, heating elements 130 may be equipped with a device that allows for checking correct operation. For example, heating elements 130 may be connected to controller 102 which may monitor proper operation of heating elements 130 and/or may control heating elements 130. In some embodiments, controller 102 may be embedded in heating breastplate 200, as shown in FIG. 4. In some embodiments, controller 102, batteries 106, and/or heating elements 130 may include battery charge indicator 131. Battery charge indicator 131 may be located on one or more batteries 106 and/or may be displayed on display device 108. Battery charge indicator 131 may include, for example, a bargraph type indicator, a light-emitting diode (LED) indicator, a percentage indicator displayed on display device 108, or other suitable type of indicator.

In some embodiments, display device 108 may be positioned on heating breastplate 200. Display device 108 may be configured to display a level of charge of batteries 106 and/or a time associated with a duration of use of the one or more batteries (e.g., 45% charge, 1 hour of charge remaining, and/or the like).

In some embodiments, when a battery level drops below a threshold battery level (e.g., and battery charge indicator 131 indicates the battery charge level is low), battery charge indicator 131 displayed on display device 108 may blink and/or flash (e.g., rapid blink 3-5 times) and/or controller 102 may trigger an audible alarm at specified time intervals when battery charge indicator is low (e.g., below 15% battery charge) and/or when controller 102 receives a signal indicating that battery charge level is low. In some embodiments, when heating element 130 powers up, battery charge indicator 131 may turn on or off in the event of a malfunction of one or more components of electrical assembly 110. In this way, it may be possible to check for correct operation of heating breastplate 200 before using heating breastplate 200 and it may also be possible to check a level of charge of batteries 106 via batter charge indicator 131.

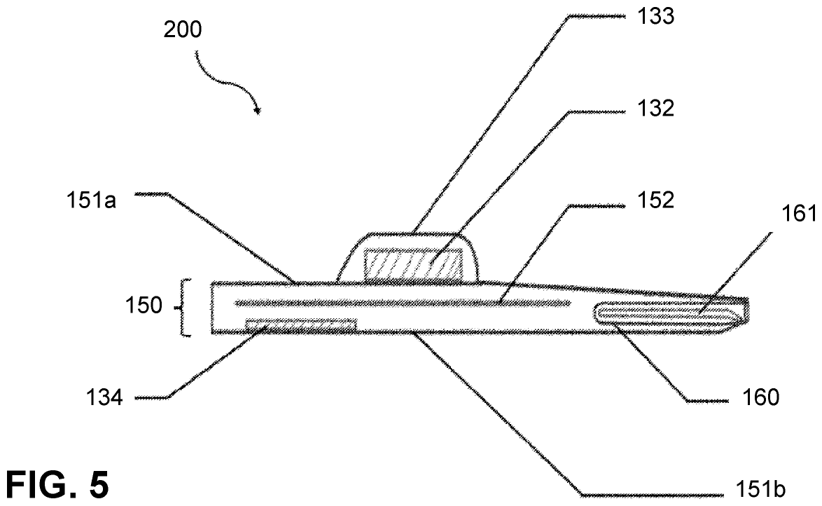

Referring to FIG. 5, FIG. 5 shows a schematic view of a heating breastplate according to section AA of FIG. 2 in accordance with non-limiting embodiments of the disclosed subject matter. Heating breastplate 200 may be composed of multilayer textile 150. Multilayer textile 150 may be formed of a first outer layer 151a corresponding to a front face of heating breastplate 200 and of a second outer layer 151b corresponding to a rear face of heating breastplate 200. Layers 151a and 151b may be sewn together in order to provide multilayer textile 150 that is thin and compact. In order to improve solidity of the multilayer textile assembly, seams may be located in areas where there is less resistance. For example, seams may be located on edges or on a central section of multilayer textile 150 with low to no resistance. Heating element 130 may be inserted as heating layer 133 in the thickness of heating breastplate 200 between the two outer layers 151a and 151b, as shown in FIG. 5.

Outer layers 151a and 151b may be sealed in order to constitute a barrier to liquids and to not allow fluids to pass through the layers. In some embodiments, it may be possible to wash outer layers 151a and 151b and clean them with decontamination products. In some embodiments, layers 151a and 151b must conform to hygiene and medical health standards and/or policies. In view of various properties, layers 151a and 151b may be polyamide coated with an anti-flammable polyurethane polymer, type M1 according to fire safety standards. Any other suitable type of material may be used as long as the material meets the need expressed and described in various medical health standards and/or policies. In some embodiments, particular treatments may be applied to layers 151a and 151b to reinforce properties of layers 151a and 151b, such as for example, a bactericidal treatment. Layers 151a and 151b may be identified by a color code in order to avoid confusion of use between the front face and the rear face of heating breastplate 200. For example, red/orange could be used on layer 151b corresponding to the rear face and blue/black may be used on layer 151a corresponding to the front face. In this way, heating breastplate may indicate which face is the face that is heated by heating elements and/or heating resistors 130.

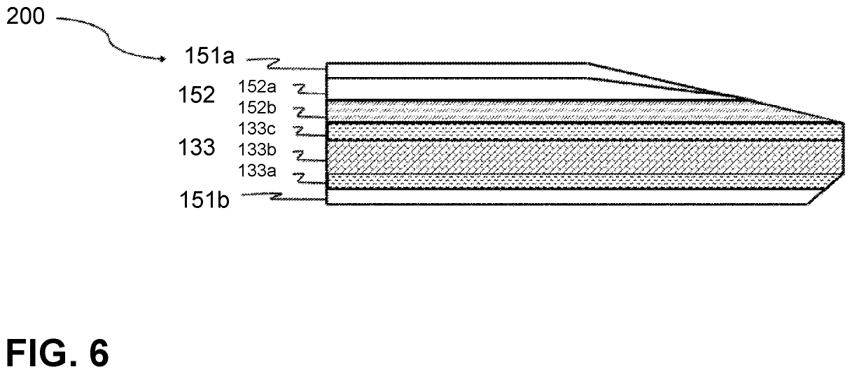

In some embodiments, multilayer textile 150 may include an additional layer in order to concentrate heat and send heat back to body 300 of the injured patient and thus improve insulation and limit heat loss. For example, multiplayer textile 150 may include an additional thermal insulation layer 152 that is located between the outer layer of the front face 151a and the heating layer 133, as illustrated in FIG. 5. In some embodiments, thermal insulation layer 152 may include a laminate of cellulose 152a and aluminum 152b as shown in FIG. 6.

According to an embodiment of heating element 130, shown in FIG. 6, for which the heating breastplate 200 may include multilayer textile 150, heating element 130 may include heating layer 133 inserted into a thickness of heating breastplate 200, as previously described and illustrated in FIG. 5. Heating layer 133 may include an electrical circuit whose electrical conductors are made in the form of a printed circuit. Such a printed circuit may include at least one insulating layer 133a covered with a conductive layer 133b on which heating resistors 130 are etched. In some embodiments, layers 133a and 133b may be flexible. Insulating layer 133a may be a polyester substrate. Conductive layer 133b may be made of aluminum. In some embodiments, in order to ensure protection of the printed circuit, conductive layer 133b may be covered by a protective layer 133c which may be bonded, for example, by hot lamination, to conductive layer 133b. Protective layer 133c may be made of flexible polyester. In some embodiments, a second protective layer 133c may also be deposited, for example by bonding, on insulating layer 133a. In some embodiments, heating breastplate 200 may include various materials such that heating breastplate 200, associated electrical assembly 110, and other components may be water resistant.

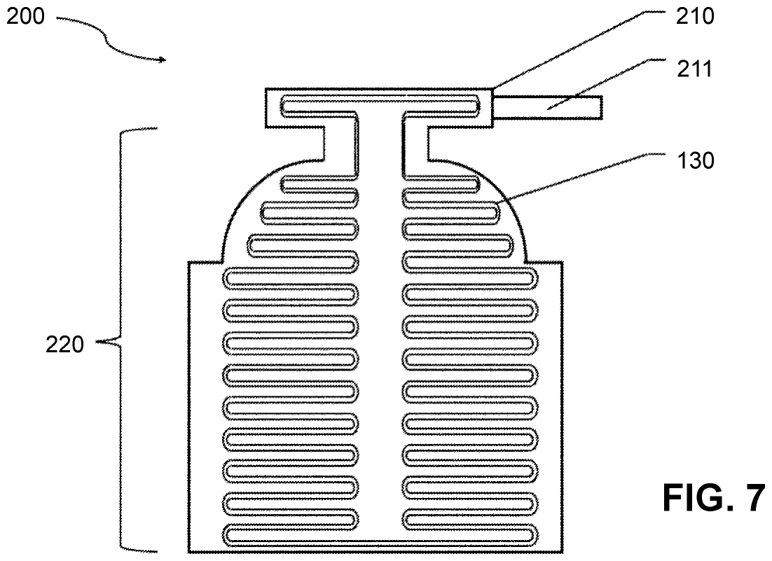

According to an embodiment of heating element 130, as shown in FIG. 7, heating element 130 may include one or more flexible electrical wires powered by a current from batteries 106. As shown in FIG. 7, a single conductor may form an overall heating resistor 130 which extends over an entire surface of the heating breastplate 200, from the neckline 210 to lower part 220. In this way, this configuration makes it possible to homogenize heating within heating breastplate 200 by using global heating. Conversely, in an embodiment described in FIG. 1, heating may be more concentrated on areas of heating breastplate 200 where the various heating resistors 131 are positioned.

In addition, heating element 130 may be connected to an electronic card ensuring regulation of temperature by continuous measurement of the current, resistance, and/or voltage. In some embodiments, measurement of current, resistance, voltage, and/or tension makes it possible to provide a measurement of resistance which is variable according to the temperature. As such, when the resistance increases, the temperature increases (e.g., a positive temperature coefficient). It may thus be possible to determine a value of the temperature from the measured value of the resistance. In this way, accidental overheating and risk of burns may be avoided by reducing a rate of heating when a targeted temperature within heating breastplate 200 is reached but also may allow for the system to trigger/accelerate heating to maintain batteries 106 above a threshold operating temperature in extreme conditions, particularly in extreme cold in external environments (e.g., avoiding deterioration and/or loss of charge of batteries 106).

In this way, heating breastplate 200 may save energy necessary such that batteries 106 do not lose charge and therefore may extend a duration of operation of heating breastplate 200. As such, power of batteries 106 may be controlled by controller 102 and electronics assembly 110 connected to heating element 130. Batteries 106 and heating element 130 may then be connected to controller 102 by cables secured by secure and sealed connectors in order to avoid any risk of disconnection and short circuit. In some embodiments, heating breastplate 200 may include an interface such as display device 108 providing temperature set point indications and/or temperature measurements, to display messages and also a level of charge of batteries 106 (e.g., battery charge indicator 131) as well as buttons providing for adjustment of a heating setpoint. An interface such as display device 108 may be located on an outer layer of the front face in an easily accessible and visible zone for the user (e.g, display device 108 as shown in FIG. 4). Display device 108 may also be connected to an audible alarm system providing audible warnings in the event of a malfunction of controller 102, heating element 130, and/or of the batteries 106, or of a low charge level of batteries 106.

Storage bag 161 may allow for storage and may protect heating breastplate 200 when heating breastplate 200 is not in use. In this way, rescuers may carry on foot heating breastplate 200 stored in storage bag 161 and/or wear storage bag 161 on a harness for interventions in dangerous environments. In order not to misplace storage bag 161, storage bag 161 may, for example, be sewn or connected by a cord to heating breastplate 200. Storage bag 161 may be made of a waterproof textile in order to protect heating breastplate 200 against weather conditions, including wet conditions. In some embodiments, storage bag 161 may be closed by a cord-type quick-tightening system, and/or the like.

Power cables 162 may allow for recharging batteries 106. Power cables 162 may be of the jack plug type, for example. The positioning of power cables 162 may make it possible to recharge batteries 106 when heating breastplate 200 is folded up and kept in storage bag 161. Recharging of batteries 106 may be performed via an external charger (e.g., charger 104), which may have a system allowing the external charger to determine a level of charge of batteries 106.

As an example, when heating breastplate 200 is folded up and/or kept in storage bag 161, power cables 162 may be connected to batteries 106 to charge batteries 106. In some embodiments, when power is on for heating element 130 and/or heating breastplate 200 when heating breastplate 200 is folded up and/or kept in storage bag 161 and power cables 162 are connected to batteries 106 to charge batteries 106, controller 102 may generate an audible alarm indicating that power of heating element 130 and/or heating breastplate 200 is on while charging batteries 106. In this way, heating breastplate 200 may prevent operator error of powering on heating breastplate 200 while batteries 106 are being charged via power cables 162 and a power source.

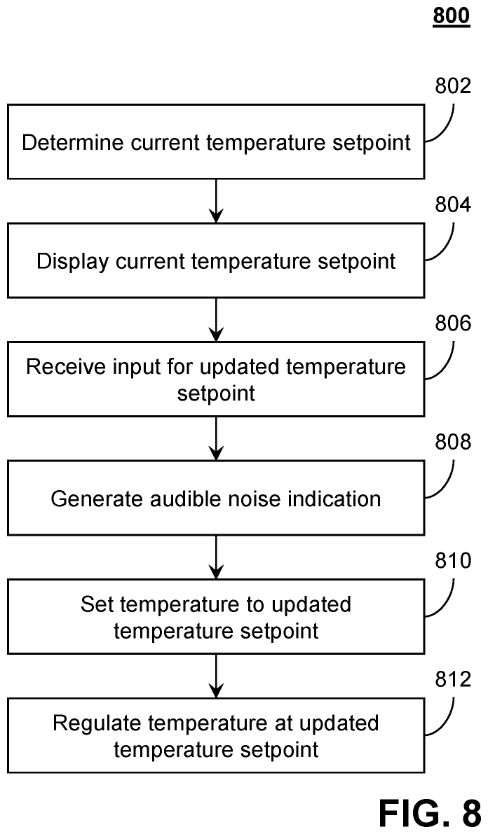

FIG. 8 shows a flow chart of a method 800 for updating a temperature of a heating element of a heating breastplate according to non-limiting embodiments of the disclosed subject matter. The steps shown in FIG. 8 are for example purposes only. It will be appreciated that additional, fewer, different, and/or a different order of steps may be used in non-limiting embodiments. Heating breastplate 200 (e.g., controller 102 thereof) may include one or more buttons to adjust a temperature setpoint of heating element 130. For example, heating breastplate 200 may include a button to adjust a temperature setpoint of heating element 130 to a warmer temperature and/or a button to adjust the temperature setpoint of heating element 130 to a cooler temperature. In some embodiments, a temperature setpoint of heating element 130 may only be adjusted and/or updated when heating breastplate 200 is powered on.

As shown in FIG. 8, at step 802, method 800 may include determining a current temperature setpoint. For example, controller 102 may determine a current temperature setpoint of heating elements 130 and/or heating breastplate 200. In some embodiments, controller 102 may have the current temperature setpoint stored in memory. In some embodiments, controller 102 may determine the current temperature setpoint by measuring a temperature of heating element 130.

As shown in FIG. 8, at step 804, method 800 may include displaying the current temperature setpoint. For example, controller 102 (e.g., display device 108 thereof) may display and/or cause display device 108 to display the current temperature setpoint. In this way, a user can view an indication of the current temperature setpoint of heating element 130 and/or heating breastplate 200 to consider whether to update the current temperature setpoint or to leave the temperature setpoint at a current temperature value.

As shown in FIG. 8, at step 806, method 800 may include receiving input for an updated temperature setpoint. For example, controller 102 may receive input for an updated temperature setpoint via one or more buttons (e.g., buttons 140) on heating breastplate 200 pressed by a user. Pressing the one or more buttons may cause a signal to be transmitted to controller 102 such that controller 102 updates and/or changes the current temperature setpoint value to an updated temperature setpoint value. For example, a current temperature setpoint may be set at a current temperature setpoint value of 40° C. A user my press a button indicating a decrease in the current temperature setpoint value by 5° C. Controller 102 may receive a signal based on a user pressing the button causing controller 102 to decrease the current temperature setpoint value by 5° C. (e.g., from 40° C. to 35° C.). Controller 102 may update the current temperature setpoint to an updated temperature setpoint. The updated temperature setpoint value may then be equal to 35° C. In some embodiments, heating element 130 and/or heating breastplate 200 may include an upper and/or a lower bound for a temperature setpoint value. For example, heating element 130 and/or heating breastplate 200 may include an upper bound for a temperature setpoint value equal to 40° C. and a lower bound for the temperature setpoint value equal to 30° C.

As shown in FIG. 8, at step 808, method 800 may include generating an audible noise indication. For example, controller 102 may generate an audible noise indication for various events including, but not limited to, pressing one or more buttons, changing a temperature setpoint value, powering on/off controller 102 and/or heating element 130, battery charge dropping below a threshold battery charge value, a malfunction in heating breastplate 200, and/or the like. The audible noise indication may be a different audible noise for different types of events. The audible noise indication may be generated by an audio component included in controller 102.

As shown in FIG. 8, at step 810, method 800 may include setting a temperature to an updated temperature setpoint. For example, controller 102 may set the temperature setpoint of heating element 130 and/or heating breastplate 200 to the updated temperature setpoint value received by controller 102 based on a user pressing one or more buttons. Controller 102 may, for example, update the current temperature setpoint value of 40° C. to the updated temperature setpoint value of 35° C.

As shown in FIG. 8, at step 812, method 800 may include regulating a temperature at the updated temperature setpoint. For example, controller 102 may regulate a temperature of heating element 130 and/or heating breastplate 200 at the updated temperature setpoint. Controller 102 may regulate a temperature of body 300 of an injured patient by regulating the temperature of heating element 130 and/or heating breastplate 200. Controller 102 may be programmed or configured to regulate the temperature of heating element 130 to provide a target temperature value of heating element 130 such that the target temperature of heating element 130 is equivalent or within an error threshold of the current and/or updated temperature setpoint. In this way, heating element 130 may provide a uniform and/or constant temperature to body 300 of the injured patient.

In some embodiments, when regulating the temperature of heating element 130, controller 102 may be further programmed or configured to determine a first temperature value of heating element 130 based on a resistance of the heating element (e.g., using a positive temperature coefficient as a reference). Controller 102 may be further programmed or configured to compare the first temperature value of heating element 130 to a temperature threshold. Controller 102 may be further programmed or configured to regulate the temperature of heating element 130 to a target temperature by increasing or decreasing the temperature of the heating element based on comparing the first temperature value of the heating element to the temperature threshold. In some embodiments, the temperature threshold may be set by a user of heating breastplate 200 via one or more inputs, such as buttons.

In some embodiments, determining a first temperature value of heating element 130 based on a resistance of heating element 130 may include measuring the resistance of the heating element to provide a resistance value. For example, controller 102 may measure a resistance value of heating element 130 while electric current is supplied to heating element 130 to determine the resistance of heating element 130. The resistance (e.g., resistance value) of heating element 130 may be used to determine a temperature of heating element 130 based on a positive temperature coefficient. In some embodiments, controller 102 may determine the first temperature value of heating element 130 by comparing the resistance value to a reference relationship of resistance and temperature based on a positive temperature coefficient.

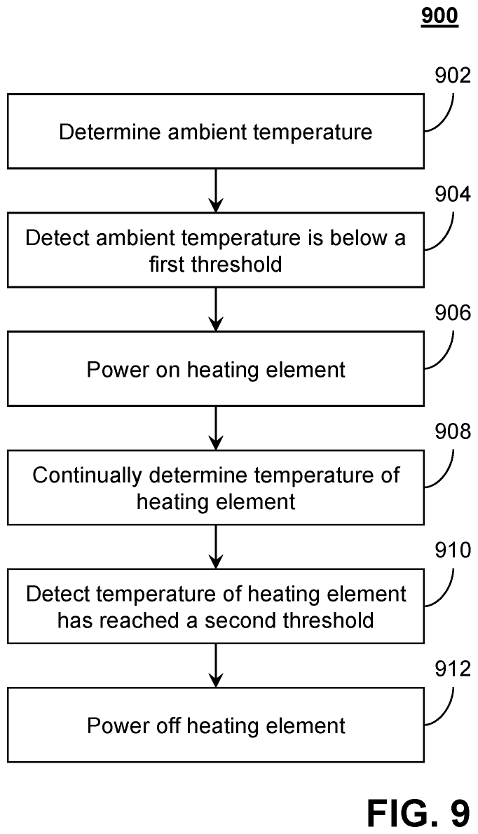

FIG. 9 shows a flow chart of a method 900 for automatically protecting a temperature of one or more batteries of a heating breastplate according to non-limiting embodiments of the disclosed subject matter. The steps shown in FIG. 9 are for example purposes only. It will be appreciated that additional, fewer, different, and/or a different order of steps may be used in non-limiting embodiments. Heating breastplate 200 (e.g., controller 102 thereof) may include automatic control to adjust a temperature of heating element 130 to maintain a temperature of batteries 106 such that batteries 106 do not deteriorate and/or suffer from a loss of charge in a harsh (e.g., cold) external environment. For example, controller 102 may include logic to automatically adjust a temperature of heating element 130 to a warmer temperature when controller 102 detects that the temperature of heating element 130 has dropped below a first temperature threshold. Once the temperature of heating element 130 has reached or exceeded a second temperature threshold, controller 102 powers off heating element 130. In some embodiments, the first temperature threshold and the second temperature threshold may be set by a user of heating breastplate 200 via one or more inputs, such as buttons. Alternatively, the first temperature threshold and the second temperature threshold may be preprogrammed into controller 102 such that the first and second temperature thresholds cannot be changed and/or set by a user.

In some embodiments, controller 102 of heating breastplate 200 may include a minimum temperature threshold such that controller 102 may regulate the temperature of heating element 130 to maintain a temperature of batteries 106 above the minimum temperature threshold.

As shown in FIG. 9, at step 902, method 900 may include determining an ambient temperature. For example, controller 102 may determine an ambient temperature via a temperature sensor arranged on heating breastplate 200 and/or by measuring a temperature of heating element 130.

As shown in FIG. 9, at step 904, method 900 may include detecting that the ambient temperature is below a first temperature threshold (e.g., a lower bound). For example, controller 102 may detect that the ambient temperature is below a first temperature threshold based on the ambient temperature determined via a temperature sensor arranged on heating breastplate 200 and/or by measuring a temperature of heating element 130. Controller 102 may compare an ambient temperature value to the first temperature threshold to detect that the ambient temperature is below the first temperature threshold.

As shown in FIG. 9, at step 906, controller 102 may power on heating element 130. For example, controller 102 may automatically power on heating element 130 based on detecting that the ambient temperature is below the first temperature threshold. In some embodiments, controller 102 may include one or more safety locks when automatically powering on heating element 130. For example, controller 102 may compare a series of measurements of ambient temperature to the first temperature threshold and detect that the ambient temperature is below the first temperature threshold using multiple ambient temperature values sequentially within a specified time before heating element 130 may be automatically powered on by controller 102.

As shown in FIG. 9, at step 908, method 900 may include continually determining a temperature of heating element 130. For example, controller 102 may continually (e.g., every few seconds, minutes, etc.) determine a temperature of heating element 130 after controller 102 has automatically powered on heating element 130 based on detecting that the ambient temperature is below the first temperature threshold.

As shown in FIG. 9, at step 910, method 900 may include detecting that the temperature of heating element 130 has reached (e.g., is at least equal to) a second temperature threshold. For example, controller 102 may detect that the temperature of heating element 130 (e.g., the temperature that is continually determined in step 908) has reached the second temperature threshold (e.g., an upper bound). Controller 102 may determine the temperature of heating element 130 based on measuring the temperature and/or resistance of heating element 130 in response to electric current flowing through heating element 130.

As shown in FIG. 9, at step 912, method 900 may include automatically powering off heating element 130. For example, controller 102 may automatically power off heating element 130 based on detecting that the temperature of heating element 130 has reached the second temperature threshold. In some embodiments, controller 102 may automatically power off heating element 130 in response to other factors and/or events including, but not limited to, detecting one or more malfunctions and/or detecting one or more operator errors.

Although embodiments have been described in detail for the purpose of illustration, it is to be understood that such detail is solely for that purpose and that the disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect.

What is claimed is:

1. A system for controlling temperature of a heating breastplate placed on a body of an injured patient, comprising:
a heating element positioned within a heating breastplate, the heating element comprising a positive temperature coefficient, the heating element connected to one or more batteries positioned within the heating breastplate; and
a controller connected to the one or more batteries and the heating element to regulate a temperature of a body of an injured patient, wherein the controller is programmed or configured to regulate the temperature of the heating element to provide a target temperature value of the heating element, such that heating element provides a uniform and constant temperature to the body of the injured patient.

2. The system of claim 1, wherein, when regulating the temperature of the heating element, the controller is further programmed or configured to:
determine a first temperature value of the heating element based on a resistance of the heating element;
compare the first temperature value of the heating element to a temperature threshold; and
regulate the temperature of the heating element to the target temperature by increasing or decreasing the temperature of the heating element based on comparing the first temperature value of the heating element to the temperature threshold.

3. The system of claim 2, wherein determining a first temperature value of the heating element based on a resistance of the heating element comprises:
measuring the resistance of the heating element to provide a resistance value; and
determining the first temperature value of the heating element by comparing the resistance value to a reference relationship of resistance and temperature based on a positive temperature coefficient.

4. The system of claim 1, wherein the one or more batteries are configured to connect to a battery charger while remaining positioned within the heating breastplate.

5. The system of claim 4, wherein the heating breastplate is placed on the body of the injured patient.

6. The system of claim 1, wherein the temperature threshold is set by a user of the heating breastplate.

7. The system of claim 1, wherein the controller of the heating breastplate comprises a minimum temperature threshold such that the controller regulates the temperature of the heating element to maintain a temperature of the one or more batteries above the minimum temperature threshold.

8. The system of claim 1, further comprising a display positioned on the heating breastplate, wherein the display is configured to display a level of charge of the one or more batteries and a time associated with a duration of use of the one or more batteries.

9. The system of claim 1, wherein the heating breastplate is water resistant.

10. A heating breastplate intended to be placed on the body of an injured patient, the breastplate comprising a neckline and a lower part respectively hugging the neckline for the neck and the thorax and the abdomen for the lower part of the breastplate, the breastplate comprising one or more heating elements arranged and positioned within the breastplate to heat the thorax, abdomen and neck of the injured patient after appropriate application of the heating breastplate to the body of the injured patient, wherein
the neckline and the lower part of the breastplate are arranged, by their respective shapes and dimensions, to be positioned and adjusted on the body of the injured patient, from the front face of the body and without mobilizing the injured patient;
the breastplate comprising one or more batteries connected to the one or more heating elements to supply electrical energy to the one or more heating elements; and
a lower third of the heating breastplate comprises one or more pockets configured to receive the one or more batteries so as to make it easier to deploy and hold the heating breastplate on the injured patient's body by gravity.

11. The heating breastplate of claim 10, further comprising a controller connected to the one or more batteries and the heating element to regulate a temperature of the body of the injured patient, wherein the controller is programmed or configured to regulate the temperature of the heating element to provide a target temperature value of the heating element, such that heating element provides a uniform and constant temperature to the body of the injured patient.

12. The heating breastplate of claim 10, wherein the one or more batteries are configured to connect to a battery charger while remaining positioned within the breastplate.

13. A heating breastplate intended to be placed on a body of an injured patient comprising a neckline and a lower part matching the body of the injured patient respectively at the level of the neck and trunk of the injured patient and a heating element for warming the body of the injured patient, wherein the heating element is positioned in the neckline and at the level of the lower part.

14. The heating breastplate according to claim 13, wherein the heating element comprises at least one flexible heating resistor formed of electrical conductors powered by batteries, the at least one flexible heating resistor configured to transform electrical energy into thermal energy.

15. The heating breastplate of claim 13, comprising a multilayer textile including a first and a second outer layer of polyamide coated with a polyurethane polymer and enclosing a heating layer, the first outer layer corresponding to a front face of the heating breastplate being in contact with an external environment and the second outer layer corresponding to a rear face of the heating breastplate being in contact with the body of the injured patient.

16. The heating breastplate of claim 13, wherein the heating layer comprises a printed circuit comprising a flexible polyester substrate covered with a flexible conductive layer of aluminum, the at least one heating resistor being etched on the aluminum layer.

17. The heating breastplate of claim 16, wherein the printed circuit is covered with a protective layer of polyester.

18. The heating breastplate of claim 15, wherein the multilayer textile comprises a thermal insulation layer between the first outer layer and the heating layer, the thermal insulation layer comprising a laminate of cellulose and aluminum.

19. The heating breastplate of claim 13, comprising pockets at the level of the lower third of the heating breastplate and distributed symmetrically on either side of a center line (M) of the heating breastplate, the pockets configured to receive the batteries.

20. The heating breastplate of claim 13, comprising a storage pocket having a closure system, a storage bag of the heating breastplate and one or more power cables for batteries, the storage pocket being positioned on a lower part of the heating breastplate and configured to contain the storage bag and the power cables.
